(19) United States
(12) Patent Application Publication
(10) Pub. No.: US 2024/0099860 A1
(43) Pub. Date: Mar. 28, 2024

(54) ANKLE SYSTEM FOR USE WITH PROSTHETIC FOOT STRUCTURE

(71) Applicant: WILLOWWOOD GLOBAL LLC, Mt. Sterling, OH (US)

(72) Inventors: Evandro M. FICANHA, Grove City, OH (US); Matthew M. WERNKE, Tampa, FL (US); Alexander W. ALBURY, Galloway, OH (US)

(73) Assignee: WILLOWWOOD GLOBAL LLC, Mt. Sterling, OH (US)

(21) Appl. No.: 18/373,498

(22) Filed: Sep. 27, 2023

Related U.S. Application Data
(60) Provisional application No. 63/410,406, filed on Sep. 27, 2022.

(51) Int. Cl. A61F 2/66

(52) U.S. Cl. CPC A61F 2/6607

(57) ABSTRACT

An ankle system for use with a prosthetic foot structure includes an outer housing with at least one through hole, the outer housing configured to receive an inner housing; the inner housing comprising an adaptor, at least one pin slot, and a base. In one embodiment, the system further comprises at least one linkage pin, the linkage pin configured to tit through the at least one pin slot; wherein the inner housing is configured to move within the outer housing from a first position to a second position along an arcuate path. The system also includes a spring body having a length, the length comprising a first end and a second end, wherein the first end is disposed within the outer housing and the second end is configured to be attached to the prosthetic foot structure.

### RELATED APPLICATIONS

This application claims the benefit of provisional U.S. patent application 63/410,406, entitled ANKLE SYSTEM FOR. USE WITH PROSTEHIC FOOT STRUCTURE, filed on Sep. 27, 2022, which is incorporated herein by reference.

### TECHNICAL FIELD

The present disclosure relates to prosthetic devices and more particularly, to an ankle system that interconnects a prosthetic foot to a prosthetic limb.

### SUMMARY OF THE INVENTION

An ankle system for use with a prosthetic foot structure is described herein. The ankle system includes an outer housing with at least one through hole, the outer housing configured to receive an inner housing including an adaptor, at least one pin slot, and a base. The system further includes at least one linkage pin, the linkage pin configured to fit through the at least one pin slot; wherein the inner housing is configured to move within the outer housing from a first position to a second position along an arcuate path; and a spring body having a length, the length comprising a first end and a second end; wherein the first end is disposed within the outer housing and the second end is configured to be attached to the prosthetic foot structure.

In one embodiment, the system may further include a spring protector;:herein the spring protector is disposed between an upper surface of the first end of the spring body and a lower surface of the base of the inner housing; and wherein a rear portion of the base of the inner housing is configured to pivot against a rear portion of the spring protector as the inner housing moves from the first position to the second position within the outer housing. In one embodiment, a bumper is attached to the spring body, wherein the bumper is capable of preventing compression of the spring body past a desired. point.

In one embodiment, the system farther comprises a moveable element disposed between a lower surface of the inner housing and an upper surface of the spring protector; wherein the moveable element is movable along a horizontal axis from a position at a rear portion of the inner housing to a position at a front portion of the :inner housing along a length of an adjustable screw, In one embodiment, the movement of the moveable element along the length of the adjustable screw is constrained by at least a first and second retaining ring disposed at a first and second end of the adjustable screw.

In another embodiment, instead of a spring protector, the system includes a pivot joint connecting, the base of the inner housing to the first end of the spring body, the pivot joint including at least one dowel pin configured to be friction fit within an opening in the base of the inner housing, an opening in the first end of the spring body, or a combination thereof.

In another embodiment, the moveable element may be at least partially disposed within a slot formed in a lower surface of the inner housing and may be moveable along a horizontal axis from a position toward a rear portion of the inner housing to a position toward a front portion. of the inner housing. The system may further include a first locking screw and a second locking screw configured to engage the moveable clement within the slot.

### BRIEF DESCRIPTION OF THE DRAWINGS

FIG. 1 is a perspective view of an embodiment of the ankle system, shown attached to a prosthetic foot;

FIG. 2 is a side elevation view of the ankle system of FIG. 1;

FIG. 3 is a front view of the ankle system of FIG. 1, shown without the prosthetic foot.,

FIG. 4 is a front view of the ankle system of FIG. 1

FIG. 5 is a rear view of the ankle system of FIG. 1, shown without. the prosthetic foot;

FIG. 6 is a rear view of the ankle system of FIG.

FIG. 7 is a side elevation view in section of the ankle system of FIG. 4, taken along line 7-7, with the ankle system positioned at maximum plantarflexion;

FIG. 8 is a side elevation view in section: of the ankle system of FIG. 4, taken along line 7-7, with the ankle system positioned. at, maximum dorsiflexion;

FIG. 9 is a side elevation view in section of the ankle system of FIG. 3, taken along line 9-9;

FIG. 10 is a side elevation view in section of the inner housing of the ankle. system of FIG. 1, shown at maximum stiffness;

FIG. 11 is a side elevation view in section of the inner housing of the ankle system of FIG. 1, shown at minimum stiffness;

FIG. 12 is a front view in section of the ankle system of FIG. 2, taken along line 12-12;

FIG. 13 is a front view in section of the ankle system of FIG. 2, taken along line 13-13;

FIG. 14 is a side elevation view in section of another embodiment of the ankle system, shown at maximum stiffness;

FIG. 15 is a side elevation view in section the ankle system of FIG. 14, shown at minimum stiffness;

FIG. 16 is a detail view in section of the ankle system of FIG. 14;

FIG. 17 is a side elevation view in section of another embodiment of the ankle system, shown at minimum stiffness;

FIG. 18 is a side elevation view in section the ankle system of FIG. 17, shown at maximum stillness;

FIG. 19 is a detail view in section of the ankle system of FIG. 17;

FIG. 20 is a side elevation view in section of another embodiment of the ankle system;

FIG. 21 is a side elevation view in section of the ankle system of FIG. 20. shown at a position with decreased stiffness:

FIG. 22 is a perspective view of one embodiment of a moveable element;

FIG. 23 is a bottom perspective view of the moveable element of FIG. 22 disposed within an inner housing;

FIG. 24 is a side elevation view in section of another embodiment of the ankle system, shown attached to a prosthetic foot;

FIG. 25 is a perspective view of the inner housing of the ankle system of FIG. 20: and

FIG. 26 is a side elevation view in section of another embodiment of the ankle system, shown attached to a prosthetic foot.

FIG. 27 is a side elevation view in section of another embodiment of the ankle system. shown attached to a prosthetic foot.

### DETAILED DESCRIPTION

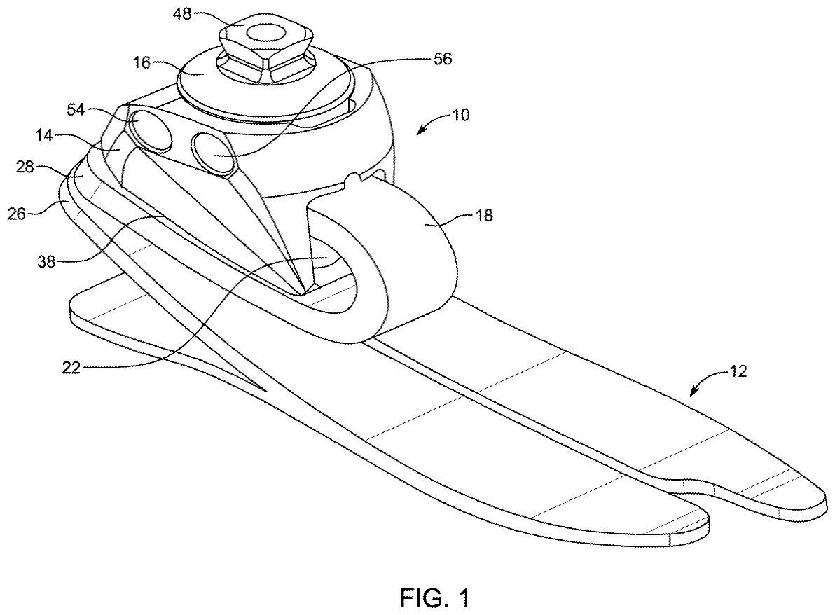

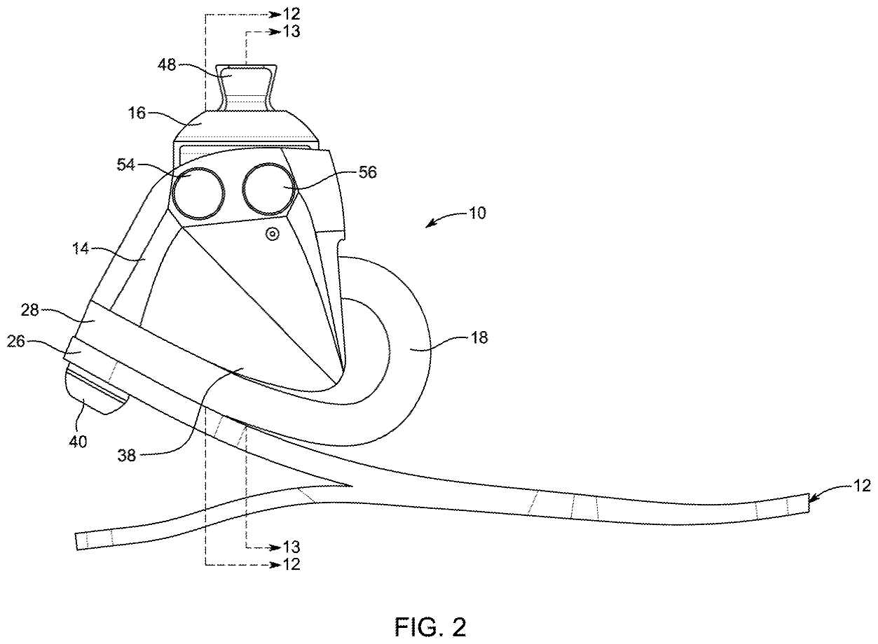

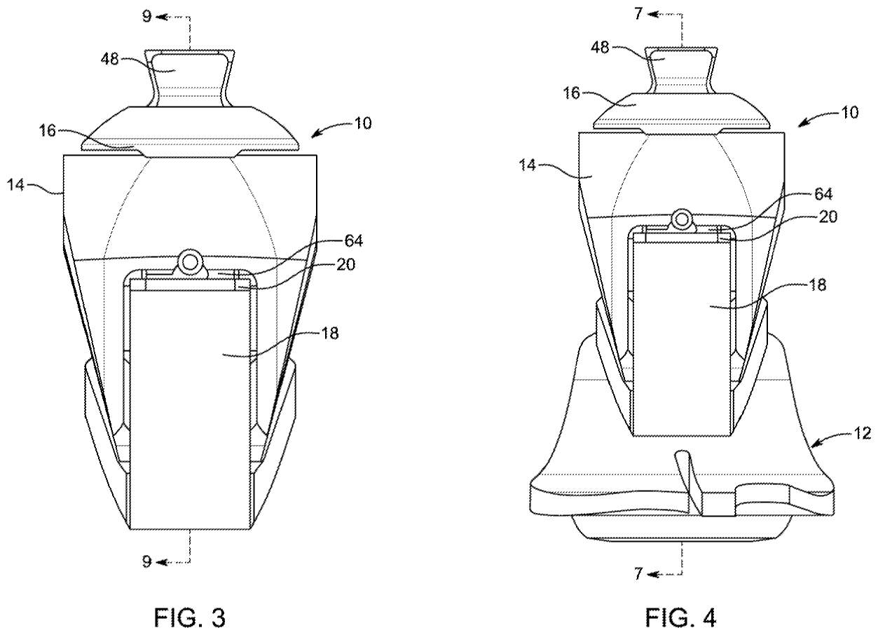

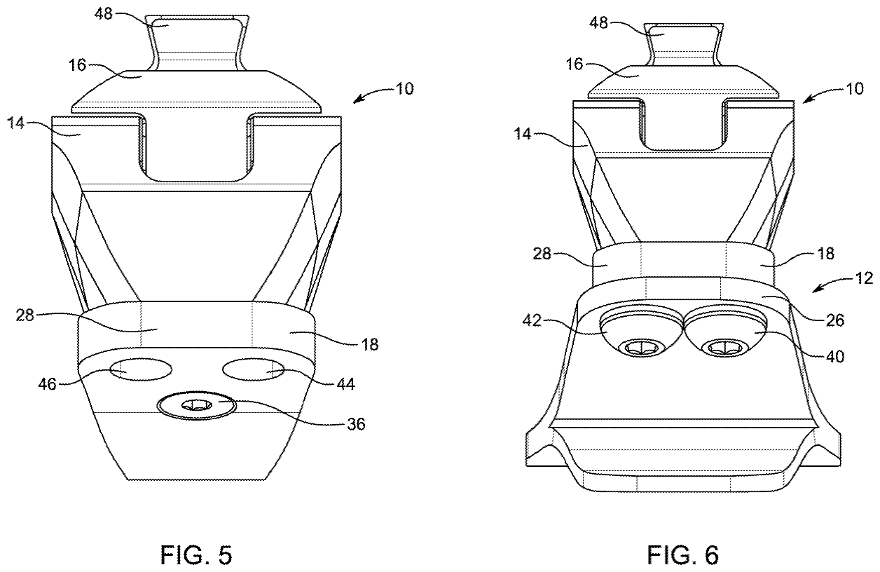

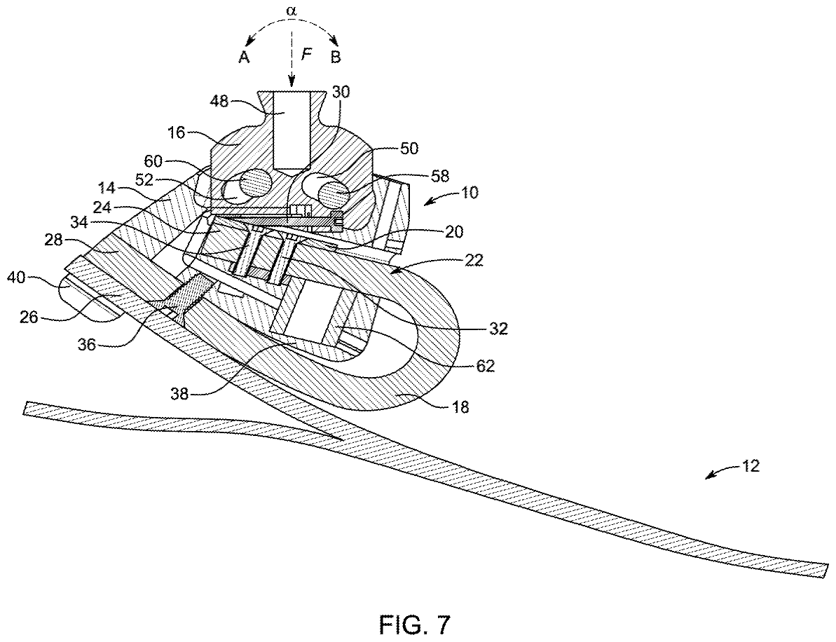

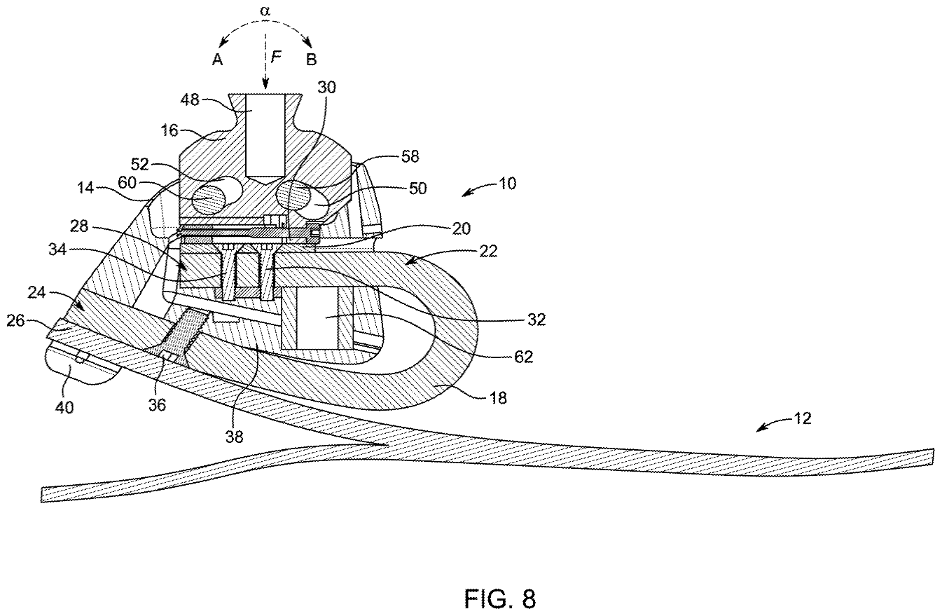

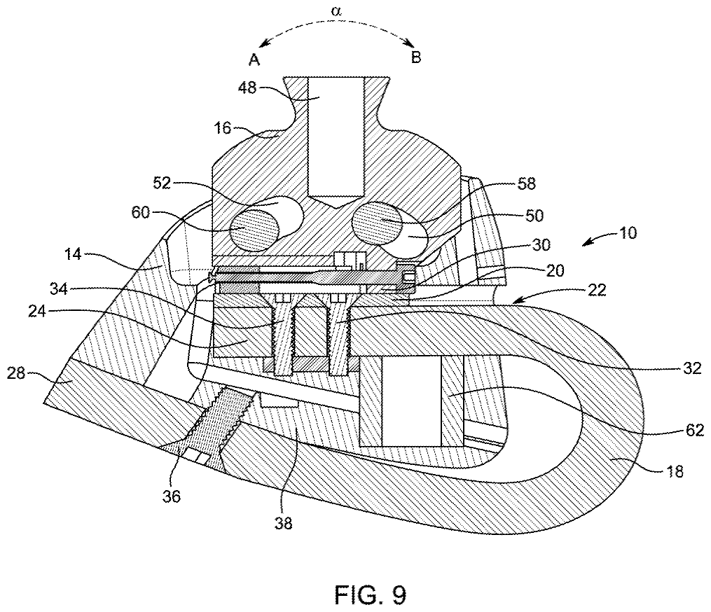

As shown in FIGS. 1-6, an embodiment of an ankle system 10 for use with a prosthesis is shown attached to a prosthetic foot 12. A prosthetic foot 12 in an embodiment may have a split toe and a heel plate and may be made of materials such as carbon fiber reinforced plastic (CFRP) or fiber glass reinforced plastic. The ankle system includes an outer housing 14, which may be made of steel, titanium, aluminum, or alloys of these, an inner housing 16, which also may be made of steel, aluminum, or titanium, or alloys of these. is received within the outer housing 14 and is capable of polycentric movement from a first position A to a second position B, along arcuate path a, relative to the outer housing 14 (as shown in FIGS. 7 and 8).

The ankle system 10 may further include at least one spring body 18 and a spring protector 20 (as shown in FIGS. 7 and 8). The spring body 18 may be received within an opening 22 in the front of the outer housing 14 at a first end 24 (as shown in FIG. 7) and attached to the heel portion 26 of the prosthetic foot 12 at a second end 28. The spring body 18 may be connected to the spring protector 20, the outer housing 14, and/or the prosthetic toot 12 at various points along the length of the spring body 18. As shown in FIG. 7, the spring body 18 is connected using a set of screws, although it should be understood that the spring body 18 may be attached using any suitable method, such as an adhesive. In this embodiment, the first end 24 is received within an opening 22 in the front of the outer housing 14 and is attached to a spring protector 20, which is disposed between the base 30 of the inner housing 16 and the spring body 18, using a first set of metal or plastic screws 32, 34 disposed through holes (not shown) within the spring protector 20. The spring body 18 is configured to extend out of the opening 22 in the front of the outer housing 14 in generally a C-shape, and to attach at a second point along its length with a second set of screws 36 (the second set 36 may include one or mores crews) to the base 38 of the outer housing 14 and at a third point (disposed at or toward the second end 28 of the spring body 18), with a third set of screws 40, 42 (see also FIG. 6) disposed through holes 44, 46 at the heel portion 26 of the prosthetic foot 12 and, optionally, holes (not shown) in the base 38 of the outer housing 14.

In one embodiment, the inner housing 16 includes a first fitting 48 that attaches to a. prosthetic leg (not shown). In an embodiment, the first fitting 48 takes the form of a male pyramid connector or adapter. As shown in FIGS. 7 and 8, the inner housing 16 also includes a plurality of curved pin slots 50, 52 disposed through the body of the inner housing (see also FIG. 15). When the inner housing 16 is disposed within the outer housing 14, at least a portion of the pin slots 50, 52 will align with through-holes 54, 56 (FIG. 1) disposed in the outer housing 14 (see also FIG. 12). Pins SS, 60 may then be inserted through both the plurality of pin slots, 50, 52 and the through-holes 54, 56 to join the outer housing 14 and the inner housing 16, while allowing the inner housing to move from a first position A to a second position B along an arcuate path a as pressure is exerted in a downward direction toward the spring body 18,

In an embodiment, the ankle system 10 further includes a stop or bumper, generally designated 62, separating the upper portion of the C-shaped spring body 18 and the base 38 of the outer housing 14. The bumper 62, which in certain embodiments is made of rubber, urethane, plastic or other compliant material, limits compression of the spring body 18 in response to the compressive force F that forces the inner housing 16 along arcuate path a into the outer housing 14. In an embodiment, the upper portion of the spring body 18 includes a recess (not shown) that receives and retains the top of the bumper 62. The bottom of the bumper 62 engages with the base 38 of the outer housing 14.

As shown in FIGS. 7 and 8, when the male pyramid connector 48 of the inner housing 16 is attached to a prosthetic. leg, such as by engaging a female pyramid connector (not shown), and the ankle system 10 is attached to the prosthetic foot 12, the weight of the user that is placed on the residual limb inserted in the prosthetic socket is transmitted through the prosthetic socket and leg through the ankle system 10 and to the prosthetic foot 22, which is on a support surface (not shown), This weight transmission exerts the compressive force F on the spring body 18 that causes the inner housing 16 to be moved within the outer housing 14. As the compressive force F is exerted against the inner housing 16, the inner housing 16 is configured to move from a first position A (FIG. 7) to a second position. B (FIG. 8). As this movement happens, the rear corner of the base 30 of the inner housing 16 is configured to pivot against the rear edge of the spring protector 20 such that the bottom surface of the base 30 of the inner housing 16 will come to a substantially parallel position against the upper surface of the spring protector 20. At the same time, the pins 58 and 60 move along arcuate path a. within the pin slots 50, 52. This motion allows the ankle system 10 to pivot from front to back in much the same way that a native ankle joint would flex during walking.

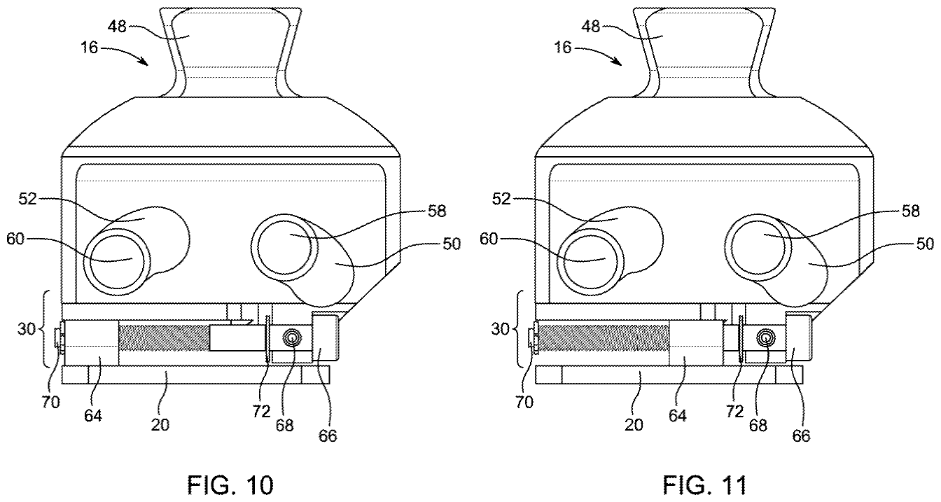

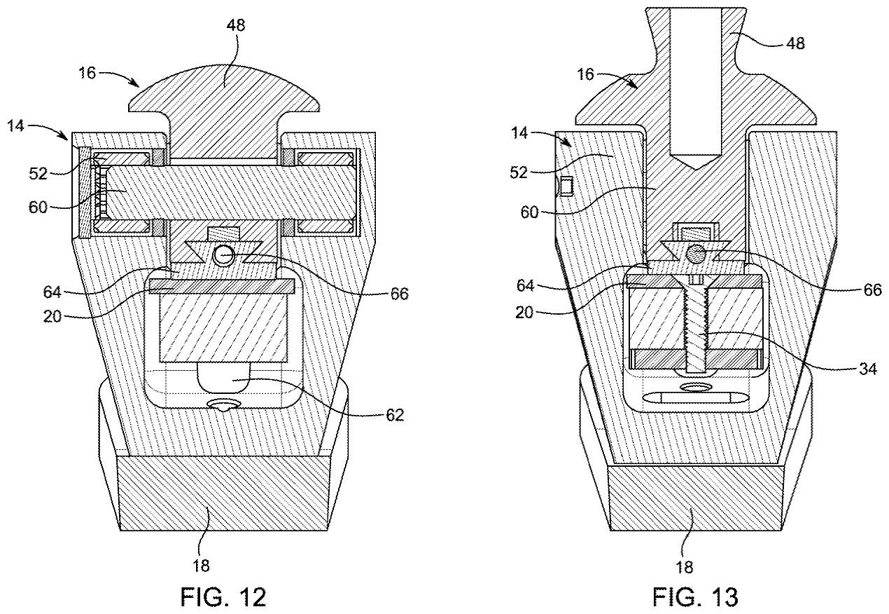

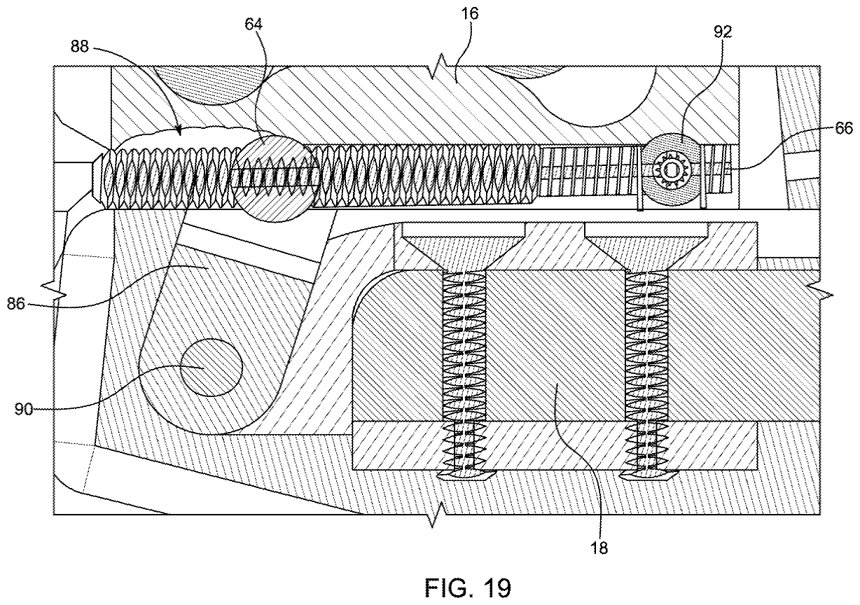

Referring now to FIGS. 10-19, the ankle system may further include an optional moveable element 64 and stiffness adjustment screw 66 disposed in the base 30 of the inner housing 16. In this embodiment, the ankle system 10 is configured to allow the user to adjust its stiffness, by changing the position at which the inner housing 16 pivots against the spring body 18 (not shown) through the spring protector 20.

In this embodiment, the motion of the pyramid adaptor 48 is transferred to the spring body 18 and bumper 62 using the moveable element 64. As shown in FIGS. 10-13, the moveable element 64 is configured to be threaded around the stiffness adjustment screw 66 such that when the adjustment screw 66 is rotated, the moveable element. 64 moves from a first position, toward the back of the inner housing base 30 (FIG. 10) to a position closer to the front of the inner housing base 30 (FIG. 11). Other methods to adjust the moveable element 64 position may be used such as adjustable pins, or slots in which the moveable element can be placed. In this embodiment, the further toward the back the moveable element is placed, the stiffer the ankle joint will he.

In addition, the movement, and thereby the adjustability, of the moveable element 64 may be limited by placing retaining rings 70, 72 at the distal and proximal ends of the stiffness adjustment screw 66. And, the position of the adjustment screw 66 may be constrained using a set screw 68 or other suitable mechanism.

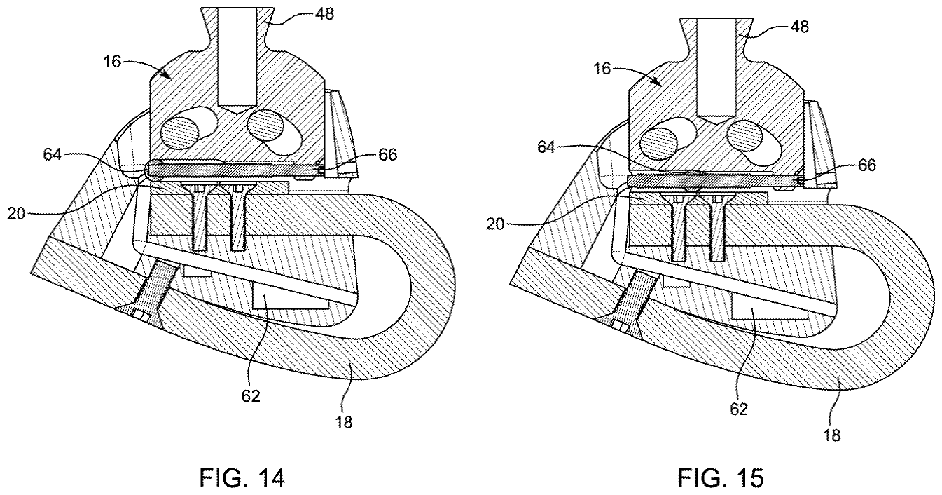

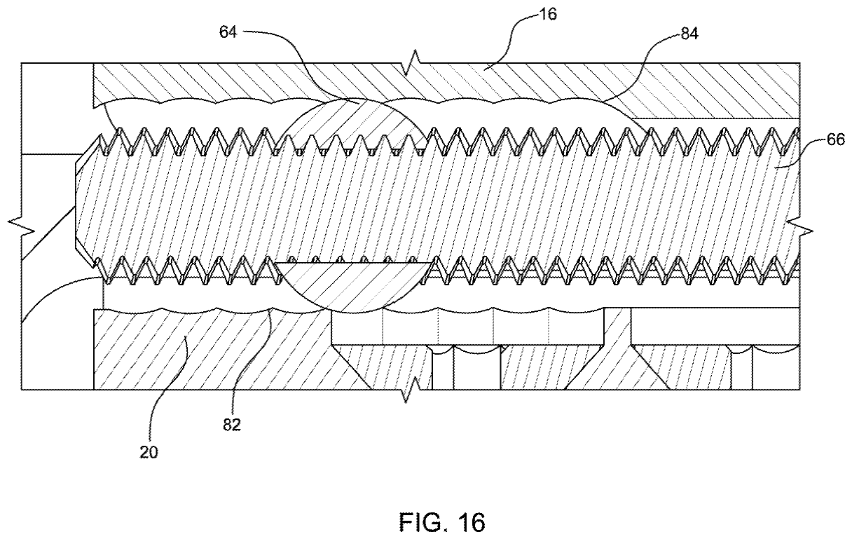

As shown in FIGS. 10 and 11, the moveable element 64 may be rectangular or, as shown in FIGS. 14-19, may be circular or round. Referring now to FIGS. 14-16, in one embodiment., the bottom surface 84 of the inner housing 16 and. the upper surface 82 of the spring protector 20 may also include grooves designed to correspond to the outer shape of the circular moveable element 64, as shown in :Fig. 16. The grooves, combined with the shape of the moveable element provide stiffness and adjustability in discreet increments.

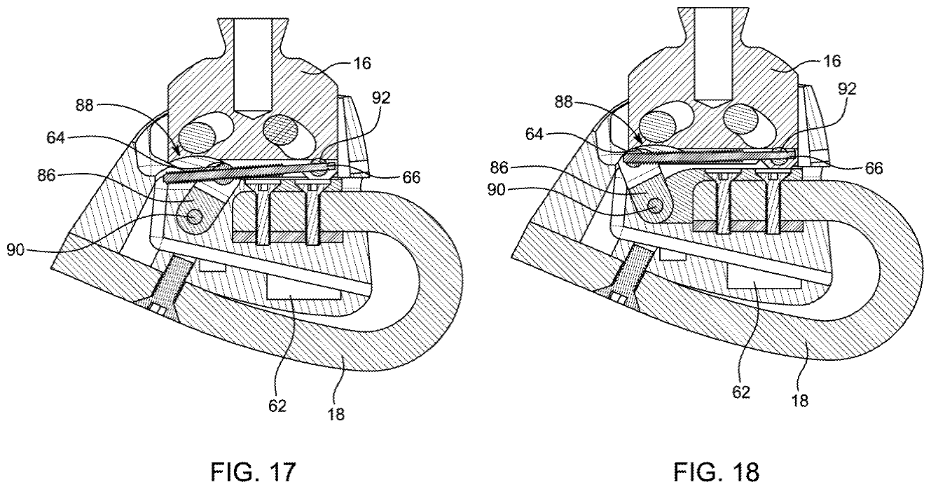

And, referring now to FIGS. 17-19, in another embodiment, the round moveable element 64 may be attached to a linkage member 86, which is configured to move from a first position to a second position, relative to the inner housing 1.6, along an arcuate smooth or grooved path $8 disposed along the bottom surface of the inner housing 16. In this embodiment, the linkage member $6 maybe attached at one end to the moveable element 64 and at a second end with a pin joint 90. In another embodiment, the adjustment screw 66 may be attached to the inner housing 16 with another pivot joint 92.

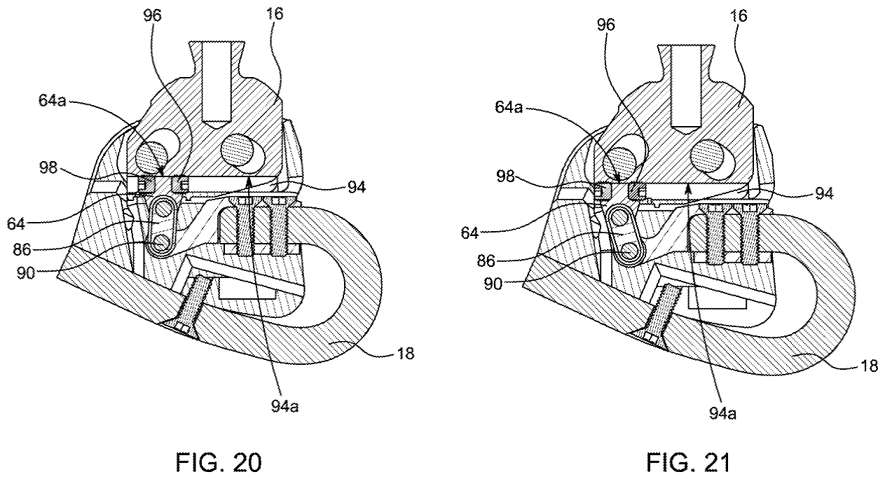

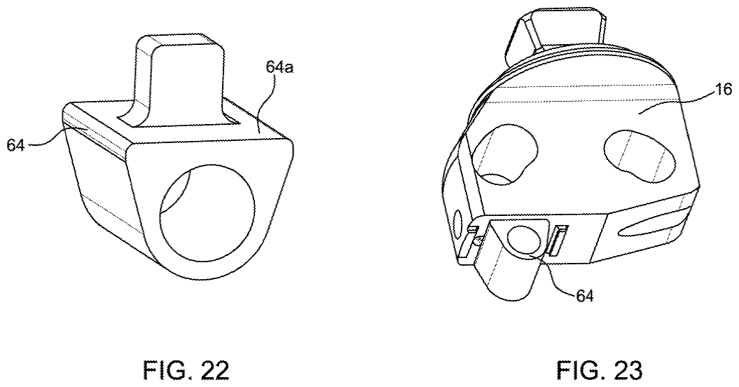

Referring now to FIG. 20, in another embodiment, the moveable element 64 is configured. to move from a first position to a second position along a straight path. In this embodiment, the movable element 64 (FIG. 22) includes a substantially flat upper surface 64a which moves within. a slot 94 disposed in the bottom surface of the inner housing 16 (FIG. 23). In this embodiment, the inner housing may include a substantially flat lower surface 94a that is configured to conform to the shape of the upper surface 64a of the moveable member 64, allowing the moveable member 64 to move forward and back within the slot 94.

Moreover, the moveable element 64 may be configured to be moved and locked in place with n the slot 94 using a pair of stiffness adjustment and/or locking screws 96 and 98. As shown in FIGS. 20 and 21, moving the screw 98 toward the back of the inner housing 16 and tightening the screw 96 toward the front of the inner housing 16 moves the movable element 64 within the slot 94 to the back and increases stiffness (FIG. 20). Similarly, loosening the screw 96 and tightening the screw 98 moves the movable element 64 to the right and decreases stiffness (FIG. 21).

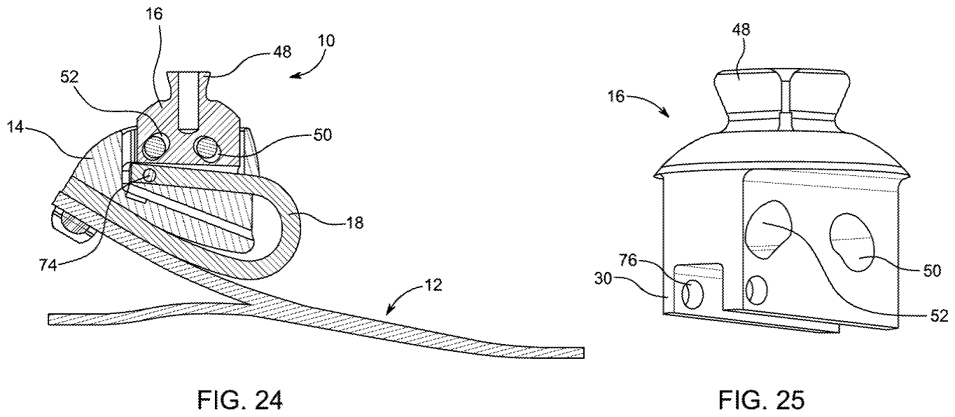

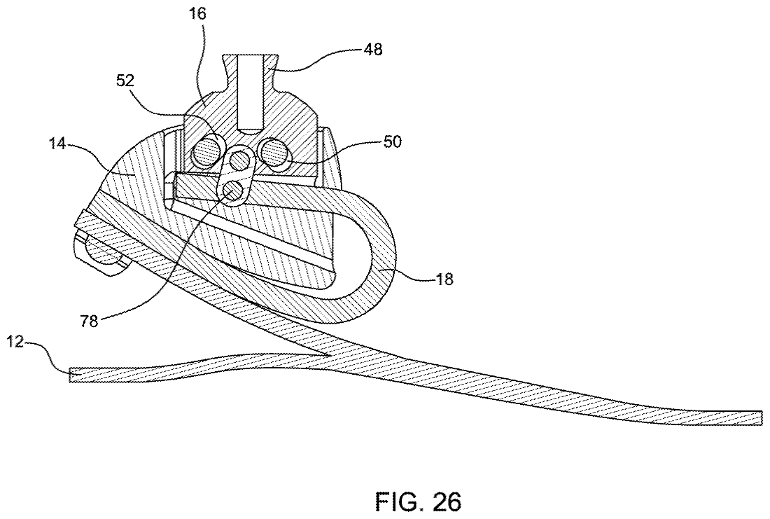

Referring now to FIGS. 24 and 25, in one embodiment, the spring body 18 is directly connected to the inner housing 16 with a pivot joint that includes at least one dowel pin. 74 inserted through a through hole 76 in the base 30 of the inner housing 16. As the ankle rotates, the motion of the inner housing 16 will transfer to the spring body 18 via the dowel pin joint, In another embodiment, as shown in FIG. 26, a rigid link member 78 connects the spring body 18 to the base 30 of the inner housing 16. The pivot joints of the rigid link member 78 allows the link to align itself between the spring body 18 and inner housing 16. The pivot joints may include dowel pins riding inside bushings and/or bearings that are press fit into each element.

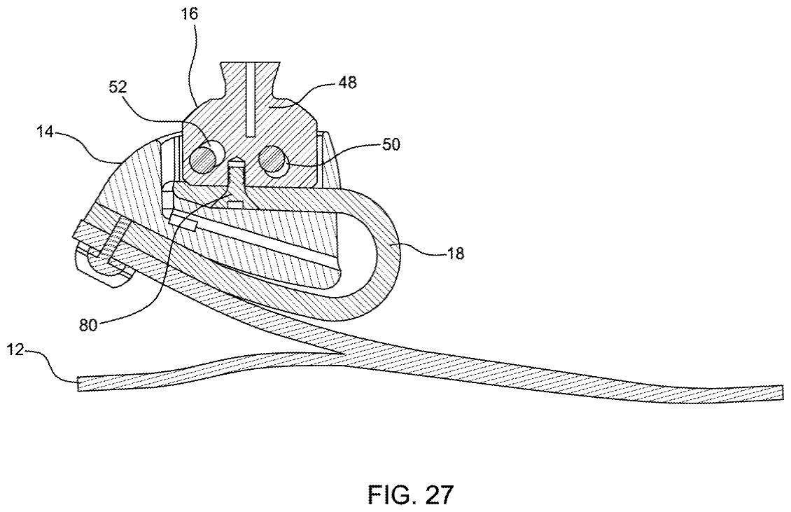

Referring now to FIG. 27, in another embodiment, the spring body 18 connects to the inner housing 16 with a fastening, mechanism, such as a bolt 80 (one or more) or some other fastener. As the ankle joint rotates, the spring body 18 is configured to bend and move with the inner housing 16, as opposed to deflecting away from the inner housing in response to contact with the spring protector. In one embodiment, the inner housing 16 and the spring body 18 may be made of separate pieces. In another embodiment, the components may be made as a single component.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, he based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another, For example, a first element could be termed a second element, and, similarly, a second element could be termed a first element, which changing the meaning of the description, so long as all occurrences of the “first element” are renamed consistently and all occurrences of the “second element” are renamed consistently. The first element and the second element are both elements, but they are not the same element.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also he understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and components, but do not preclude the presence or addition of one or more other futures, integers, steps, operations, elements, components, and or groups thereof.

As used herein, the term “if” may be construed to mean “when” or “upon” or “in response to determining or “in accordance with a determination” or response to detecting,” that a stated condition precedent is true, depending on the context. Similarly, the phrase “if it is determined. [that a stated condition precedent is true]” or “if [a stated condition precedent is truer]”or “when [a stated condition precedent is true]” may he construed to mean “upon determining” or “in response to determining” “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the .implementations shown and described herein are only illustrative of the principles of the present invention and that various modifications may be implemented by those skilled in the art without departing from the scope and spirit of the invention.

What is claimed is:

1. An ankle system for use with a prosthetic foot structure, the ankle system comprising:
an outer housing comprising at. least one through hole, the outer housing configured to receive an inner housing;
the inner housing comprising an adaptor, at least one pin slot, and a base;
at leas one linkage pin, the linkage pm configured to fit through the at least one pin slot;
wherein the inner housing is configured to move within the outer housing from a
first position to a second position along an arcuate path; and
a spring body having a length, the length comprising a first end and a. second end: wherein the first end is configured to engage the inner housing and the second end is configured to engage the prosthetic foot structure.

2. The ankle system of claim 1, wherein the system further comprises a spring protector;
wherein the spring protector is disposed between an upper surface of the first end of the spring body and an lower surface of the base of the inner housing; and wherein a rear portion of the base of the inner housings is configured to pivot against a rear portion of the spring protector as the inner housing moves from the first position to the second position within the outer housing.

3. The ankle system of claim 1, wherein the inner housing comprises at least a second pin slot and the system further comprises a second linkage pin; wherein the pin slots are disposed within inner housing along the arcuate path.

4. The ankle system of claim 1, wherein the inner housing comprises a. plurality of pin slots and the system further comprises a corresponding number of linkage pins; wherein the plurality of pin slots are disposed within inner housing along the arcuate path..

5. The ankle system of claim 1, wherein the system further comprises a moveable element disposed between a lower surface of the inner housing and an upper surface of the spring; and
wherein the moveable element is movable along a horizontal axis from a position at a rear portion of the inner housing to a position at a front portion of the inner housing.

6. The ankle system of claim wherein the moveable element is configured to move along an adjustable screw.

7. The ankle system of claim. 6, wherein movement of the moveable element along the adjustable screw is constrained by at least a first and. second. retaining ring disposed at a first and second end of the adjustable screw.

8. The ankle system of claim 1, wherein the system fin her comprises a moveable element, wherein the moveable element is at least partially disposed within a slot formed in a lower surface of the inner housing; and wherein the moveable element is movable along a horizontal axis from a position toward a rear portion of the inner housing to a position toward a front portion of the inner housing.

9. The ankle system of claim wherein he system further comprises a first locking screw and a second locking screw configured to engage the .moveable element within the slot.

10. The ankle system: of claim I, wherein the system further comprises a pivot joint connecting the base of the inner housing. to the first end of the spring body, the pivot joint comprising at least one dowel pin configured to be fit within an opening in the base of the inner housing, an opening in the first end of the spring body, or a combination thereof.

11. The ankle system of claim 1, wherein the base of the inner housing is attached directly to the first end of the spring body.

12. The ankle system of claim 1, wherein the system further comprises a bumper attached to the spring body.
